(19) United States
(12) Patent Application Publication
(10) Pub. No.: US 2002/0115073 A1
(43) Pub. Date: Aug. 22, 2002

(54) Genome-based personalized medicine

(76) Inventors: Nickolas Papadopoulos, Brookline, MA (US); Hai Yan, Baltimore, MD (US); Bert Vogelstein, Baltimore, MD (US); Kenneth W. Kinzler, Bel Air, MD (US)

Correspondence Address: BANNER & WITCOFF, 1001 G STREET N W, SUITE 1100, WASHINGTON, DC, 20001, US

(21) Appl. No.: 09/784,305

(22) Filed: Feb. 16, 2001

(51) Int. Cl. C12Q 1/68; C12N 15/02

(52) U.S. Cl. 435/6; 435/449

(57) ABSTRACT

Individual alleles can be isolated from every chromosome within somatic cell hybrids generated from a single fusion event. Nucleic acids or proteins from the hybrids can be analyzed for polymorphisms to provide unambiguous determinations. Information thus obtained can be used to develop and implement personalized medical interventions for individuals having particular polymorphic markers.

This invention was supported with U.S. government funds, NIH grants CA43460, CA57345, CA62924, CA67409, CA72851. The government therefore retains certain rights in the invention.

### BACKGROUND OF THE INVENTION

The science of pharmacogenomics uses information about genetic variation in populations to predict drug responses. Kleyn & Vesell, Science 281, 1820-21, 1998. The science of pharmacogenetics, on the other hand, uses an individual's genetic information to predict drug responses in that individual. Bullock, Drug Benefit Trends 11, 53-54, 1999. With the sequencing of the human genome nearing completion, it will become more and more commonplace to identify genetic mutations which cause a disorder, which predispose an individual to a disorder, or which may affect an individual's response to a drug and then to tailor a medical intervention for that individual.

Accurate identification of polymorphic markers is essential for this individualized approach to therapy. The problem with humans and other mammals, however, at least from a genetic diagnostic perspective, is that they are diploid. For example, mutations in one allele, such as those responsible for all dominantly inherited syndromes, are always accompanied by the wild-type sequence of the second allele. Though many powerful techniques for genetic diagnosis have been developed over the past decade, all are compromised by the presence of diploidy in the template. For example, the presence of a wild-type band of the same electrophoretic mobility as a mutant band can complicate interpretation of sequencing ladders, especially when the mutant band is of lower intensity. Deletions of a segment of DNA are even more problematic, as in such cases only the wild-type allele is amplified and analyzed by standard techniques. These issues present difficulties for the diagnosis of monogenic diseases and are even more problematic for multigenic diseases, where causative mutations can occur in any of several different genes. Such multigenism is the rule rather than the exception for common predisposition syndromes, such as those associated with breast and colon cancer, blindness, and hematologic, neurological, and cardiovascular diseases. The sensitivity of genetic diagnostics for these diseases is currently suboptimal, with 30% to 70% of cases refractory to genetic analysis.

There is a need in the art for a method for simply separating and analyzing individual alleles from human and other mammalian cells, so that an individual's genetic profile can be accurately obtained and individualized medical interventions can be determined and implemented based on that genetic profile.

### SUMMARY OF THE INVENTION

It is an object of the invention to provide methods of personalizing medical interventions for individual patients. This and other objects of the invention are provided by one or more of the embodiments described below.

One embodiment of the invention is a method of identifying a personalized medical intervention for a non-rodent individual predisposed to or having a disorder associated with at least one polymorphic marker in at least one gene or in at least one intergenic region. Cells of the non-rodent individual are fused to rodent cell recipients to form non-rodent/rodent cell hybrids. Fused cell hybrids are selected for by selecting for a first selectable marker contained on a rodent chromosome and for a second selectable marker contained on a first non-rodent individual chromosome, to form a population of fused cell hybrids. A subset of hybrids which are haploid for a second non-rodent individual chromosome which is not the same chromosome as the first non-rodent individual chromosome and which was not selected is detected among the population of fused cell hybrids. Said subset of hybrids is analyzed to detect a polymorphic marker in the gene, in a product of the gene, or in the intergenic region, wherein the gene or intergenic region resides on the second non-rodent individual chromosome. A medical intervention is selected based on identity of the gene or intergenic region.

Another embodiment of the invention is a method of identifying a non-rodent individual as eligible to participate in a clinical trial to study the efficacy of a medical intervention. Cells of the non-rodent individual are fused to rodent cell recipients to form non-rodent/rodent cell hybrids. Fused cell hybrids are selected by selecting for a first selectable marker contained on a rodent chromosome and for a second selectable marker contained on a first non-rodent chromosome. A population of fused cell hybrids is formed. A subset of hybrids which are haploid for a second non-rodent chromosome which is not the same chromosome as the first non-rodent chromosome and which was not selected is detected among the population of fused cell hybrids. The subset of hybrids is analyzed to detect a polymorphic marker in a gene, in a product of the gene, or in the intergenic region, wherein the gene or intergenic region resides on the second non-rodent chromosome. The non-rodent individual is identified as eligible to participate in the clinical trial based on the presence, absence, or identity of third polymorphic marker which is detected.

Yet another embodiment of the invention is a method of identifying a polymorphic marker as associated with a first subpopulation of non-rodent individuals. Cells of a plurality of non-rodent individuals are fused to rodent cell recipients to form a plurality of non-rodent/rodent cell hybrids. Fused cell hybrids are selected by selecting for a first selectable marker contained on a rodent chromosome and for a second selectable marker contained on a first non-rodent chromosome. A population of fused cell hybrids is formed. A subset of hybrids which are haploid for a second non-rodent chromosome which is not the same chromosome as the first non-rodent chromosome and which was not selected is detected among the population of fused cell hybrids. The subset of hybrids is analyzed to detect a polymorphic marker in the gene, in a product of the gene, or in the intergenic region, wherein the gene or intergenic region resides on the second non-rodent chromosome. The polymorphic marker is identified as associated with the first subpopulation if the polymorphic marker is more prevalent in the first subpopulation and if the polymorphic marker is less prevalent in a second subpopulation of non-rodent individuals.

Still another embodiment of the invention is a method of identifying a diagnostic test to be performed on a non-rodent individual predisposed to or having a disorder associated with at least one polymorphic marker in at least one gene or in at least one intergenic region. Cells of the non-rodent individual are fused to rodent cell recipients to form non-rodent/rodent cell hybrids. Fused cell hybrids are selected for by selecting for a first selectable marker contained on a rodent chromosome and for a second selectable marker contained on a first non-rodent individual chromosome. A population of fused cell hybrids is formed. A subset of hybrids which are haploid for a second non-rodent individual chromosome which is not the same chromosome as the first non-rodent individual chromosome and which was not selected is detected among the population of fused cell hybrids. Said subset of hybrids is analyzed to detect a polymorphic marker in the gene, in a product of the gene, or in the intergenic region, wherein the gene or intergenic region resides on the second non-rodent individual chromosome. A diagnostic test is performed based on the presence, absence, or identity of the polymorphic marker which is detected.

The invention thus provides methods of identifying and implementing personalized medical interventions and diagnostic tests, optimizing the usefulness of clinical trials, and of identifying polymorphic markers which predispose or cause a particular disorder.

### BRIEF DESCRIPTION OF THE FIGURES

FIG. 1. Strategy for hybrid generation. The recipient mouse cell line E2 was fused with human lymphocytes and clones were subsequently selected with HAT plus geneticin, which kill unfused E2 cells and lymphocytes, respectively. All clones contained a human X chromosome responsible for growth in HAT. Clones were genotyped to determine which human chromosomes were retained. Chromosomes marked “A” and “B” represent the two homologs of a representative human chromosome. The average proportion of clones which retained neither, both, or either of the six chromosome homologs analyzed is indicated (see text). Mutational analysis was carried out on nucleic acids of clones which retained single alleles of the genes to be tested.

FIG. 2. Allelic status and gene expression in hybrids. (FIG. 2A) Polymorphic markers from the indicated chromosomes were used to determine the genotype of the indicated hybrids. “Donor” denotes the human lymphocytes used for fusion with the mouse recipient cells. (FIG. 2B) cDNA of E2 and four hybrids were used as templates to amplify hMSH2, hMSH6, hMLH1, hTGF TGF-β-RII, hPMS1, hPMS2, and APC sequences. The results were concordant with the genotypes observed in (FIG. 2A), in that hybrids 5-7 retained at least one allele of each of the chromosomes containing the tested genes, while hybrid 8 contained alleles of chromosomes 3, 5, and 7 but not of chromosome 2 (containing the hMSH2, hPMS1, and hMSH6 genes).

FIG. 3. Mutational analysis of an HNPCC patient refractory to standard genetic diagnosis. Nucleic acids from the indicated hybrids were tested for retention of chromosomes 2 and 3 using polymorphic markers (FIG. 3A) and for expression of hMSH2 and hMLH1 genes on chromosomes 2 and 3, respectively (FIG. 3B). Hybrids 1, 2, 3, and 6 contained allele A from chromosome 2 and did not express hMSH2 transcripts, while hybrids 4 and 5 contained the B allele and expressed hMSH2. hMLH1 expression served as a control for the integrity of the cDNA. (FIG. 3C) Sequences representing the indicated exons of hMSH2 were amplified from the indicated hybrids. Exons 1-6 were not present in the hybrids containing allele A, but exons 7-16 were present in hybrids containing either allele.

FIG. 4. Mutational analysis of Warthin family G. (FIG. 4A) Sequence analysis of RT-PCR products from hMSH2 transcripts of hybrid 1, containing the mutant allele of a Warthin family G patient, illustrates a 24 bp insertion (underlined; antisense primer used for sequencing). The wild-type sequence was found in hybrid 3, containing the wt allele. RT-PCR analysis of transcripts from lymphoid cells of the patient showed that the mutant transcript was expressed at significantly lower levels than the wild-type sequence. Sequence analysis of the genomic DNA of the same hybrids (FIG. 4B) showed that the insertion was due to an A to C mutation (antisense sequence, indicated in bold and underlined) at the splice acceptor site of exon 4, resulting in the use of a cryptic splice site 24 bp upstream. The signal of the mutant C is not as strong as the wild-type A in the donor's DNA. Such non-equivalence is not unusual in sequencing templates from diploid cells, and can result in difficulties in interpretation of the chromatograms. (FIG. 4C) Extracts from hybrids 1 and 5, carrying the mutant allele of chromosome 2, were devoid of hMSH2 protein, while extracts of hybrids 2 and 3, carrying the wt allele, contained hMSH2 protein. Hybrid 4 did not contain either allele of chromosome 2. Hybrids 1, 3, 4, and 5 each carried at least one allele of chromosome 3 and all synthesized hMLH1 protein. β-tubulin served as a protein loading control. Immunoblots with antibodies to the indicated proteins are shown.

FIG. 5. Schematic view of genome-based personalized medicine.

### DETAILED DESCRIPTION

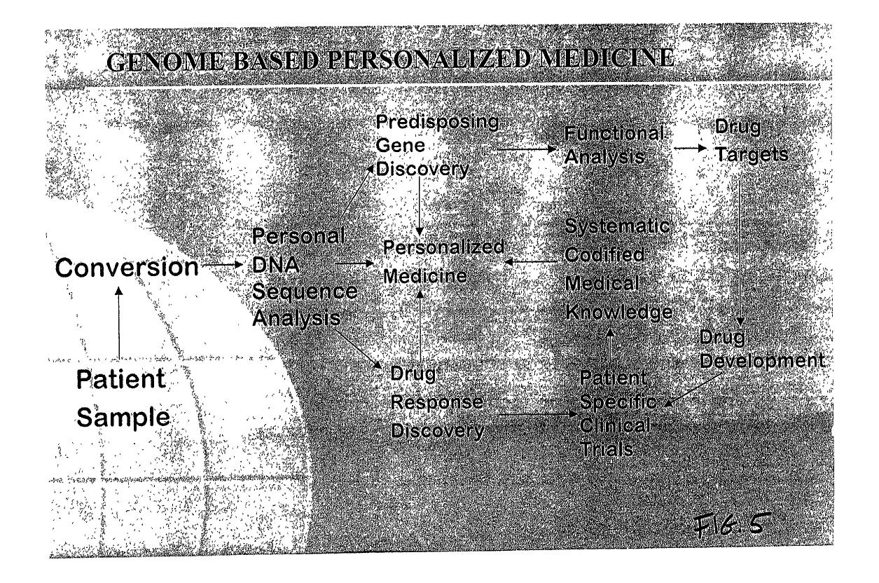

It is a discovery of the present invention that information provided by the improved accuracy of genetic diagnosis obtained through the use of the non-rodent/rodent cell hybrids described below can be used to develop individual DNA sequence profiles, drug response profiles, functional response profiles, protein profiles, and personalized medical interventions, as well as drugs designed to interact with particular target molecules. For example, an individual's risk of developing disorders such as heart disease, diabetes, or cancer can be simply and accurately determined and an appropriate therapeutic regimen prescribed. In addition, an individual's likely response to a particular therapeutic agent can be determined and an appropriate dosage regimen identified. Accurate genetic diagnosis using the disclosed cell hybrids also can be used to identify genes that cause or predispose an individual to a disorder and to identify individuals as qualified to participate in clinical trials. See FIG. 5.

Generation of Non-rodent/rodent Cell Hybrids

We have devised a strategy for generating hybrids containing any desired human or other mammal's chromosome using a single or multiple fusion and selection conditions. Importantly and unexpectedly, the human or other mammalian chromosomes in these hybrids are stable, and they express human or other mammalian genes at levels sufficient for detailed analysis. The approach is based on the principle that fusion between human or other mammal and rodent cells creates hybrid cells that contain the full rodent genomic complement but only a portion of the human or other mammalian chromosomes. In the past, selection for retention of a specific human or other mammalian chromosome (by complementation of an auxotrophic rodent cell, for example) has allowed the isolation of hybrids containing a desired chromosome (7, 8). Though such fusions have proven useful for a variety of purposes (8, 9), their utility has been limited by the availability of appropriate rodent recipients for many chromosomes and by the inefficiencies and variation of the fusion and selection conditions. For the analysis of multigenic diseases, it would be necessary to perform a separate fusion and selection for each chromosome.

The stability of the human or other mammalian chromosomes in the hybrids of the present invention was surprising. Though the human genetic constitution of radiation hybrids is relatively stable, this stability has been presumed to be due to the integration of small pieces of human DNA into rodent chromosomes following irradiation of the donor cells. The human chromosomes in whole cell fusions have been believed to be unstable unless continuous selection pressure for individual chromosomes was exerted. The reasons for the stability in our experiments is unclear, but may be related to the diploid nature of the rodent partner. Such diploidy reflects a chromosome stability that is unusual among transformed rodent cells. Previous experiments have indeed shown that chromosomally stable human cells retain all chromosomes upon fusion with other chromosomally stable human cells, unlike the situation when one of the two partners is chromosomally unstable.

The diploid, rodent recipient cells of the present invention provide useful reagents for the facile creation of cells with functionally haploid human or other non-rodent mammalian genomes. Nucleic acids or proteins from these hybrids can be used as reagents for any standard assay for detecting mutations or other polymorphic markers. As such assays are constantly being improved and automated (1), the value of the hybrid-generated materials correspondingly increases. It is possible, in fact, to examine the sequence of entire genes (promoters and introns in addition to exons), as well as intergenic regions. Nucleic acid templates generated from single alleles are clearly superior for such analyses, as the homogeneous nature of the templates dramatically enhances the signal to noise ratio of virtually any diagnostic assay. We therefore envision that this approach can be productively applied to a wide variety of research and clinical problems because of its power to detect polymorphic markers in genes as well as intergenic regions. Polymorphic markers include, without limitation, single nucleotide polymorphisms, microsatellite markers, mutations, and haplotypes (i.e., sets of polymorphic markers present on a single chromosome), as well as alterations in proteins, such as altered structure, function, molecular weight, amino acid sequence, etc.

Genes of interest are typically those that have been found to be involved in inherited diseases. These include genes involved in colon cancer, breast cancer, Li-Fraumeni disease, cystic fibrosis, neurofibromatosis type 2, von Hippel-Lindau disease, as well as others. The identified genes include APC, merlin, CF, VHL, hMSH2, p53, hPMS2, hMLH1, BRCA1, as well as others. Polymorphic markers which can be identified at the protein level include those in sequences that regulate transcription or translation, nonsense mutations, splice site alterations, translocations, deletions, and insertions, or any other changes that result in substantial reduction of the full-length protein or in altered expression or activity levels of the protein. Other subtler polymorphic markers can be detected at the nucleic acid level, such as by sequencing of RT-PCR products.

Cells of the human which may be used in fusions are any which can be readily fused to rodent cells. Peripheral blood lymphocytes (PBL) which are readily available clinical specimens are good fusion partners, with or without prior mitogenetic stimulation, whether used fresh or stored for over one year at −80° C. Any cells of a mammalian body can be used, because all such cells contain essentially the same genetic complement. Cells of mammals which can be used include in particular those of primates (e.g., humans, gorillas, chimpanzees, baboons, squirrel monkeys), companion animals (e.g., cats, rabbits, dogs, horses), farm animals (e.g., cows, sheep, swine, goats, horses), and research animals (e.g., cats, dogs, rabbits, sheep, goats, swine, chimpanzees, and baboons). More generically, the cells of the other mammals can be selected from the ruminants, primates, carnivora, lagomorpha, and perissodactyla. Typically the other mammalian cell fusion partner is not a rodent cell.

Rodent cell recipients for fusion are preferably diploid, more preferably oncogene-transformed, and even more preferably have microsatellite instability due to a defect in a mismatch repair gene. Selection of particular clones which grow robustly, are stably diploid, and fuse at a high rate is well within the skill of the ordinary artisan. The rodent cells may be, for example, from mice, rats, guinea pigs, or hamsters.

Fusion of cells according to the present invention can be accomplished according to any means known in the art. Known techniques for inducing fusion include polyethylene glycol-mediated fusion, Sendai virus-mediated fusion, and electro-fusion. Cells can desirably be mixed at a ratio of between 10:1 and 1:10 human to rodent. Clones of fused cells generally become visible after about two to three weeks of growth.

Fused hybrid cells can be selected using any marker which results in a positively selectable phenotype. These include antibiotic resistance genes, toxic metabolite resistance genes, prototrophic markers, etc. The surprising advantage of the present invention is that a single selectable marker on a single human or other mammalian chromosome can be used in the selection and that stable hybrids containing more than just the single, selected human or other mammalian chromosome result. Thus, polymorphic markers on other chromosomes can be analyzed even when the chromosomes on which the polymorphic markers reside were not selected.

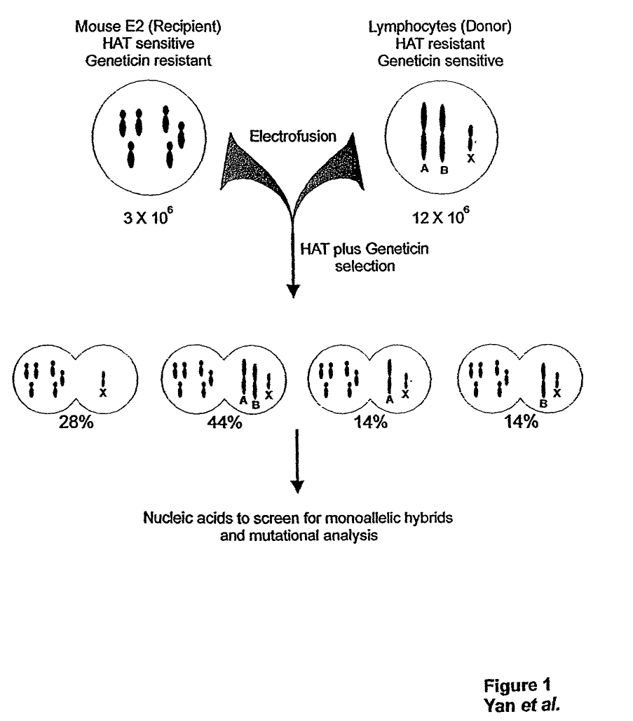

Fused hybrid cells can be analyzed to determine that they do in fact carry a human or other mammalian (non-rodent) chromosome which carries a gene of interest. Hybrid cells which have either of the two relevant human or other mammalian chromosomes can be distinguished from each other as well as from hybrids which contain both of the two human or other mammalian chromosomes. See FIG. 1. While any means known in the art for identifying the human or other mammalian chromosomes can be used, a facile analysis can be performed by assessing microsatellite markers on the human or other mammalian chromosome. Other linked polymorphic markers can be used to identify a desired human or other mammalian chromosome in the hybrids.

Once hybrid cells are isolated which contain one copy of a human or other mammalian gene or intergenic region of interest from a human or other mammal who is being tested, polymorphic marker analysis can be performed on the hybrid cells. Any portion of a DNA molecule—i.e., genes (including coding regions, regulatory elements, and untranslated regions) and intergenic regions—can be analyzed. Genes or intergenic regions can be tested directly for at least one polymorphic marker (“a polymorphic marker”). mRNA or protein products of the genes can be tested. Mutations that result in reduced expression of the full-length protein product should be detectable by Western blotting using appropriate antibodies. Tests which rely on a function of the protein encoded by a gene of interest and enzyme assays can also be performed to detect mutations. Other immunological techniques can also be employed, as are known in the art. One or more polymorphic markers can be detected, and the polymorphic markers can be located on one or more chromosomes.

If an immunological method is used to detect the protein product of a gene of interest in the hybrids, it is desirable that antibodies be used that do not cross-react with rodent proteins. Alternatively, the rodent genes which are homologous to the gene of interest can be inactivated by mutation to simplify the analysis of protein products. Such mutations can be achieved by targeted mutagenesis methods, as is well known in the art.

Functional tests can also be used to assess the normalcy of each allelic product. For example, if one inserted an expression construct comprising a β-galactosidase gene downstream from a p53 transcriptional activation site into a rodent-human hybrid cell that contained human chromosome 17 but no endogenous p53, then one could detect mutations of the p53 on the human chromosome 17 by staining clones with X-gal. Other enzymatic or functional assays can be designed specifically tailored to a particular gene of interest.

Any method of detecting polymorphic markers at the DNA or RNA level that is known in the art may be employed. These include, without limitation, sequencing, allele-specific PCR, allele-specific hybridization, microarrays, DGGE, and automated sequencing. Methods of detecting alterations at the protein level include, without limitation, non-denaturing polyacrylamide gel electrophoresis, protein activity assays (e.g., enzyme activity, ligand binding), immunological methods, cytochemistry, histological methods, and the like.

It is a possibility that expression of a gene of interest might be inhibited in the hybrid cell environment. In order for the loss of expression of a gene of interest in the hybrid cells to be meaningfully interpreted as indicating a polymorphic marker in the human or other mammal, one must confirm that the gene of interest, when wild-type, is expressed in rodent-human or other mammal hybrid cells. This confirmation need not be done for each patient, but can be done once when the assay is being established.

When the assay of the present invention indicates that a polymorphic marker exists in the gene or intergenic region of interest, other family members can be tested to ascertain whether they, too, carry the polymorphic marker. Alternatively, the other family members can be tested to see if they carry the same chromosome as the affected family member. This can be determined by testing for a haplotype, i.e., a set of distinctive markers which are found on the chromosome carrying the mutation in the affected family member. Determination of a haplotype is a by-product of performing the assay of the invention on the first family member. When the hybrid cells are tested to confirm the presence of the relevant chromosome in the hybrid, for example by use of microsatellite markers, a distinctive marker set will be identified, which can then be used as a haplotype. These haplotypes can be experimentally (i.e., directly) determined.

Mixed populations of hybrid cells made by the fusion process of the present invention may contain hybrid cells which are haploid for a number of different human or other mammalian chromosomes. Typically each homolog of at least 2, at least 5, at least 10, at least 15, at least 20, or even 22 human or other mammalian autosomes will be present in the population in a haploid condition in at least one out of one hundred, seventy-five, fifty, thirty or twenty-eight of the cells. Thus a high proportion of the cells contain multiple human or other mammalian chromosomes, and a relatively small number of cells must be tested to find cells harboring a single copy of a non-selected chromosome.

Populations of cells resulting from a single hybrid are uniform and homogeneous due to the high stability of the human or other mammalian chromosomes in the hybrid cells of the invention. Thus at least 75%, 80%, 85%, 90%, 95%, 97%, 99%, or 100% of the cells in the population resulting from a single hybrid cell contain the same complement of human or other mammalian chromosomes.

Identifying a Personalized Medical Intervention or Diagnostic Test

The use of non-rodent/rodent hybrid cells described above can be used to identify one or more polymorphic markers in one or more genes or intergenic regions which, for example, predispose an individual to a particular disorder or are causally related to a particular disorder. Mutations in genes which encode proteins that interact with a drug useful to treat a particular disorder also can be identified. Disorders include, without limitation, neoplastic diseases (including both benign and malignant tumors), nervous system disorders (including neurodegenerative disorders such as multiple sclerosis, Wilson's disease, Alzheimer's disease, Pick's disease, Huntington's chorea, Parkinson's disease, and amyotrophic lateral sclerosis; psychiatric disorders such as schizophrenia and depression; and ophthalmic disorders), deficiency disorders (including deficiencies of fat- and water-soluble vitamins and enzymes), obesity, pancreatic disorders (e.g., diabetes), respiratory disorders (e.g., chronic obstructive pulmonary disease, cystic fibrosis), liver and biliary disorders (cirrhosis, glycogen accumulation, amyloidosis, drug-induced injury and cholestasis, hepatitis), hematological disorders (e.g., hemophilia, anemias, polycythemia, thrombocytopenia, thrombocytosis), gastrointestinal disorders (e.g., esophagitis, cholitis, ulcerations, diverticulosis, scleroderma), kidney disorders (including diseases of the glomeruli, tubules, interstitium, and blood vessels, as well as obstructive and calculous nephropathy), muscle disorders (e.g., muscular atrophy, muscular dystrophy, myasthenia gravis), bone disorders (e.g., osteoporosis, dyschondroplasia, achondroplasia, Marfan's syndrome, osteopetrosis, gargoylism, Paget's disease, fibrous dysplasia), cardiovascular disorders (e.g., arteriosclerosis, Raynaud's disease, thrombophlebitis, congestive heart failure, coronary artery disease, hypertension), diseases of immunity (e.g., autoimmune disorders such as diabetes, rheumatoid arthritis, autoimmune hemolytic anemia, chronic thyroiditis, systemic lupus erythematosus, polyarteritis nodosa, polymyositis, dermatomyositis, systemic sclerosis, Sjögren's syndrome, Wegener's granulomatosis, as well as immunologic deficiency syndromes, such as alymphocytic agammaglobulinemia, Good's syndrome, thymic aplasia, infantile agammaglobulinemia, Wiskott-Aldrich syndrome, and acquired immune deficits), urinary disorders (e.g., ureteritis), endocrine disorders (e.g., congenital adrenal hypoplasia, Cushing's syndrome, primary hyperaldosteronism, Addison's disease), disorders of the reproductive system (e.g., hypospadias, epispadias, phimosis, benign prostatic hypertrophy or hyperplasia, functional abnormalities of the ovary or endometrium), connective tissue disorders (e.g., arthritis, including suppurative arthritis, tuberculous arthritis, rheumatoid arthritis, and osteoarthritis, bursitis, tenosynovitis, nodular fascitis, chordoma), skin disorders (e.g., metabolic diseases, inflammations, acne, warts, psoriasis, contact dermatitis, eczema), and infectious diseases (e.g., disorders caused by infectious agents such as viruses, bacteria, protozoa, prions, fungi, and mycoplasma).

Once a polymorphic marker has been identified, a medical intervention can be selected based on the identity of the gene or intergenic region in which the polymorphic marker resides. For example, individuals can be sorted into subpopulations according to their genotype. Genotype-specific drug therapies can then be prescribed. Medical interventions include interventions that are widely practiced, as well as less conventional interventions. Thus, medical interventions include, but are not limited to, surgical procedures, administration of particular drugs or dosages of particular drugs (e.g., small molecules, bioengineered proteins, and gene-based drugs such as antisense oligonucleotides, ribozymes, gene replacements, and DNA- or RNA-based vaccines), including FDA-approved drugs, FDA-approved drugs used for off-label purposes, and experimental agents. Other medical interventions include nutritional therapy, holistic regimens, acupuncture, meditation, electrical or magnetic stimulation, osteopathic remedies, chiropractic treatments, naturopathic treatments, and exercise.

In one embodiment, knowledge of an individual's genetic profile can be used to improve targeting of a drug to individuals who are responsive to the drug and therefore most likely to benefit from that drug. For example, metastatic breast cancer patients who overexpress HER2 can be identified and treated with HERCEPTIN®^{.} Baselge et al., Cancer Res. 58, 2825-31, 1998; Goldenberg, Clin. Therapeut. 21, 309-18, 1999. Identification of particular polymorphic markers can be used to predict the onset of a disorder, as well as to identify interventions likely to be effective to prevent or delay the onset of the disorder (i.e., prophylactic interventions) or to treat its symptoms. As used herein, “treat” includes reducing the severity or frequency of one or more symptoms as well as elimination of the symptom(s). It is known that genetic variations in apolipoprotein E can be used to identify individuals likely to develop Alzheimer's disease, as well as those who would benefit from particular interventions, such as tacrine therapy. This therapy is beneficial to patients who lack the two copies of the apoplipoprotein E4 (ApoE4) gene, whereas patients with the ApoE4 gene subtype are less responsive to tacrine therapy. Tanne, BMJ 316, 1930, 1998. Using methods of the invention, individuals likely to be resistant to a particular intervention, (including those who are non-responsive as well as those who are less responsive than other individuals) can be identified and alternative interventions prescribed for those patients.

The risk of drug toxicity and other adverse side-effects also can be minimized by more accurate genetic identification of individuals likely to suffer such effects from a particular drug. For example, breast cancer patients who have a deficiency in dihydropyrimidine dehydrogenase can develop serious neurotoxicity when treated with fluorouracil. In such patients, other drugs or dosages could be prescribed. Alternatively, the patient can be monitored for early signs of adverse side effects, and appropriate ameliorating intervention can be instituted.

In addition, prevention of unnecessary exposure to therapeutic agents that would not be effective in a particular individual can be achieved. For example, patients who lack the enzyme cytochrome CYP2D6 cannot metabolize tricyclic antidepressants (e.g., desipramine) or selective serotonin reuptake inhibitors (e.g., fluoxetine, sertraline). Bullock, 1999; Tanaka & Hisawa, J. Clin. Pharm. Ther. 24, 7-16, 1999. Individuals who produce an inactive version of the enzyme thiopurine methyltransferase (TMPT) cannot metabolize azathioprine, which is used to treat a variety of disorders, including Crohn's disease. Columbel et al., Gastroenterology 2000 June;118(6):1025-30. A single nucleotide polymorphism (SNP) exists which prevents metabolism of pravastatin, which is used to lower cholesterol. Campbell et al., Drug Discovery Today 5, 388-96, 2000. Many other pharmacologically relevant polymorphisms are well known. Id. Alternative interventions less likely to produce side-effects can be prescribed for these patients.

Accurate genetic diagnosis of polymorphic markers in a gene or intergenic region which affect peptides, proteins, or other factors involved in the efficacy or bioavailability of drugs is especially useful for identifying an appropriate medical intervention. For example, after a drug is administered, its efficacy and bioavailability depend on numerous proteins with which it interacts, including carrier proteins, metabolizing enzymes, receptors, and transporters. Sadee, Pharm. Res. 15, 959-63, 1998; Evans & Relling, Science 286, 487-91, 1999; Sadee, B. Med. J. 319, 1286, 1999; Mancinelli et al., 2000. Such proteins affect the drug's absorption, distribution, metabolism, and excretion. Variations in the enzymes that metabolize a particular therapeutic agent can affect the effective level of the therapeutic agent. It is well known that the activities or levels of various drug-metabolizing enzymes, such as acetyltransferases and sulfotransferases, exhibit genetic polymorphisms. Bullock, 1999. The principal drug metabolizing enzymes are the cytochrome P450 enzymes (e.g., CYP2D6, 3A4/3A5, 1A2, 2E1, 2C9, and 2C19). Mancinelli et al., AAPS Pharmsci 2, article 4, 2000. Cytochrome P450 enzymes (CYPs) can both activate (for example, convert codeine to morphine) and deactivate (for example, nicotine to cotinine) drugs.

Differences in drug responses due to genetic differences in proteins that interact with the drugs are well known. Up to a 16-fold variation in plasma levels of phenytoin, an anticonvulsant drug, have been observed in patients who have received the same doses of the drug. This difference is due, at least in part, to the different levels of CYP2D6 in these patients. Bullock, 1999. CYP2C19, which is involved in the metabolism of anxiolytics, such as diazepam, and anti-ulcer drugs, such as omeprazole, is polymorphically expressed. Sagar et al, Gastroenterology 2000 September;119(3):670-6. Thus, accurate knowledge of the presence of particular polymorphic markers in an individual can be used to determine appropriate doses of a drug. In addition, if expression levels of particular enzymes are known, those levels can be manipulated to increase the efficacy of a particular drug.

Accurate determination of an individual's genetic profile can also eliminate unnecessary diagnostic tests and identify those diagnostic tests which could or should be performed. Identification and selection of those diagnostic tests most likely to be performed can result in a significant savings in time and cost and can avoid unnecessary stress to the patient.

Clinical Trials

Variability between individuals can be a complicating factor in the design of clinical trials designed to study the efficacy of a known or potential medical intervention. According to another embodiment of the invention, non-rodent individuals can be identified as qualified to participate in the clinical trial or can be stratified (i.e., sorted into subgroups) based on their genetic profiles for one or more genes or intergenic regions. If desired, individuals can be qualified or stratified based, for example, on the presence, absence, or identity of polymorphic marker which is detected in a gene encoding a target of a known or potential therapeutic agent, an enzyme involved in metabolizing the known or potential therapeutic agent, or a carrier or transporter protein for the known or potential therapeutic agent.

For example, Long QT Syndrome can be caused by mutations in a number of different genes. Vincent, Cardiol Clin 2000 May;18(2):309-25; Chiang & Roden, J Am Coll Cardiol 2000 July;36(1):1-12; Allen, Nurs Clin North Am 2000 September;35(3):653-62 Similarly, subtypes of genes in the renin-angiotensin system have been associated with an increased risk of in-stent restenosis. Bauters et al., Semin Interv Cardiol 1999 September;4(3):145-9. Individuals with the same disorder but with different polymorphic markers can be sorted into subgroups according to the particular polymorphic marker(s) present in each individual. Specific therapies can then be tested in these subpopulations. Benhorin et al., Circulation Apr. 11, 2000; 101(14):1698-706. Genes which predispose an individual to a disorder, or which are causally related to a disorder, also can be tested for the presence of polymorphic markers and the individuals qualified or stratified according to the results. Thus, clinical trials can be optimized to provide useful results.

Identifying Genes Associated with Subpopulations

Polymorphic markers associated with subpopulations of non-rodent individuals can be more quickly and accurately identified using the non-rodent/rodent cell hybrid technique described herein. Cells of a plurality of non-rodent individuals can be fused with rodent cells, as described above. A polymorphic marker can be identified as associated with a particular subpopulation if the polymorphic marker is more prevalent in that subpopulation and is less prevalent in another subpopulation. Polymorphic markers can be associated with any subpopulation, including, but not limited to, ethnic subpopulations, subpopulations of individuals having a disorder, and kindreds.

In one embodiment, polymorphic markers associated with a disorder in the subpopulation can be identified. Optionally, transcription of a gene in which the polymorphic marker is identified or a function of a protein product of the gene can be assayed. Methods of assaying transcription, including cell-based and in vitro transcription assays, are well known. Assays for protein function also are well known in the art and include yeast two- and three-hybrid assays, protein binding assays, enzyme assays, and the like.

All patents and patent applications cited in this disclosure are expressly incorporated herein by reference. The above disclosure generally describes the present invention. A more complete understanding can be obtained by reference to the following specific examples, which are provided for purposes of illustration only and are not intended to limit the scope of the invention.

### EXAMPLE 1

An outline of the approach to creating non-rodent/rodent cell hybrids is presented in FIG. 1. The rodent fusion partner was a line derived from mouse embryonic fibroblasts transformed with ras and adenovirus E1A oncogenes. HPRT-deficient subclones of this line were generated, and one subclone (E2) was chosen for further experimentation based on its robust growth characteristics, maintenance of diploidy, and fusion efficiency (10). Human lymphocytes cells were mixed with E2 cells at an optimum ratio and electrofused, and hybrids selected in geneticin (to kill unfused human cells) and HAT (to kill unfused E2 cells) (11). Colonies appearing after two weeks of growth were expanded and RNA and DNA prepared for analysis. From a single fusion experiment, an average of 36 hybrid clones were obtained (range of 17 to 80 in five different individuals).

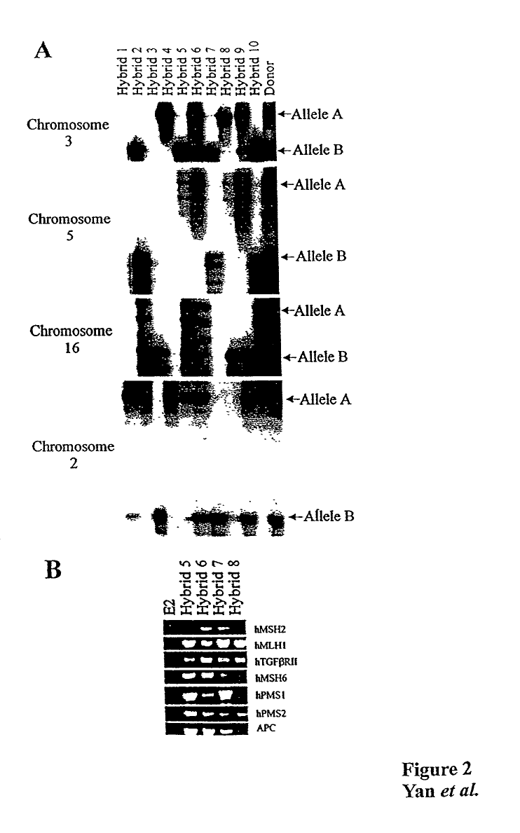

All hybrids contained the human X chromosome, as this chromosome contains the HPRT gene allowing growth in HAT. To determine whether other human chromosomes were present in the hybrids, polymorphic microsatellite markers (12) were used as probes in PCR-based assays (FIG. 2A). We focused on the chromosome arms (2p, 2q, 3p, 5q, 7q, and 16q) known to contain colorectal cancer (CRC) predisposition genes. One copy of each of these chromosome arms was present in a significant fraction of the hybrid clones. For example, of 476 hybrids derived from 14 individuals and examined for chromosome 3, 136 hybrids contained neither donor chromosome, 211 hybrids contained both donor chromosomes, 60 hybrids contained one parent's chromosome, and 69 hybrids contained the other parent's chromosome. Similar retention frequencies were found for all six chromosome arms analyzed. Testing of markers from both arms of a single chromosome showed that whole chromosomes, rather than chromosome fragments, were generally retained in the hybrids. This result was confirmed with fluorescence in situ hybridization (FISH) on metaphase spreads from the hybrids, which indicated the presence of 11±13 human chromosomes in each hybrid cell. Calculations based on the genotypic data indicated that the analysis of 25 hybrids would ensure a 95% probability of identifying at least one hybrid containing the maternal allele and one hybrid containing the paternal allele of a single chromosome under study. Moreover, it would require only 45 hybrids to similarly ensure that each allele of all 22 autosomes was present and separated from its homolog in at least one hybrid (13).

### EXAMPLE 2

Two other features of the hybrids were essential for the analyses described below. First, the human chromosome complements of the hybrids were remarkably stable. Polymorphic marker analysis in ten hybrids revealed identical patterns of retention after growth for 90 (30 passages) generations after initial genotyping. Second, those hybrids containing the relevant chromosome expressed every human gene assessed, including all known colorectal cancer susceptibility genes (the hMSH2 and hMSH6 genes on chromosome 2p, the hPMS1 gene on chromosome 2q, the TGF-β Receptor Type II gene and hMLH1 gene on chromosome 3p, the APC gene on chromosome 5q, the hPMS2 gene on chromosome 7q, and the E-cadherin gene on chromosome 16q; representative examples in FIG. 2B) (14).

### EXAMPLE 3

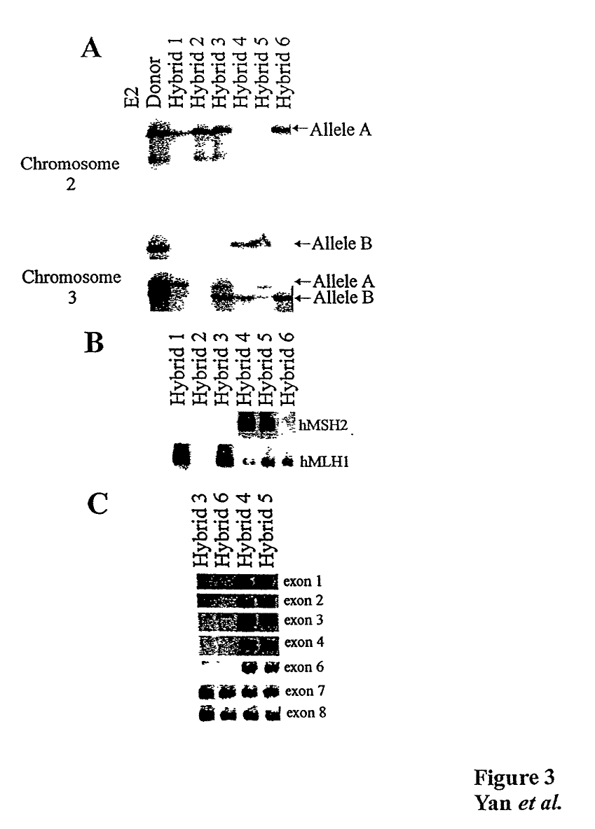

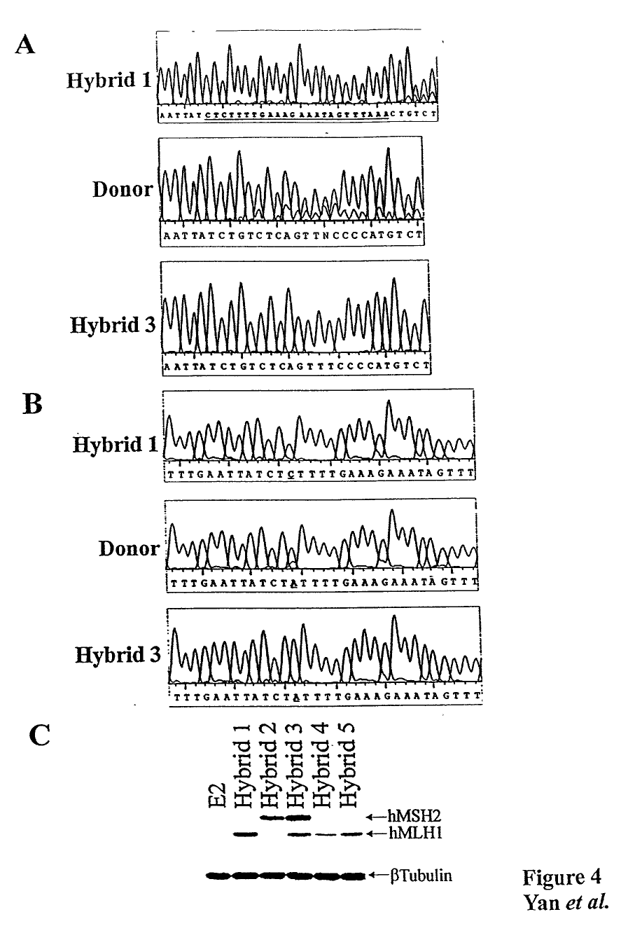

Having established the stability and expression patterns of CRC-predisposition genes in these hybrids, we used this “conversion” approach to investigate ten patients who had proven refractory to standard genetic diagnostic techniques. Each of these patients had a significant family history of colorectal cancer and evidence of mismatch repair deficiency in their tumors, yet sequencing of the entire coding sequence of each known MMR gene had failed to reveal mutations. Indeed, these and similar studies have prompted the speculation that other major HNPCC genes must exist. (25-34) Hybrids were generated from lymphocytes of each patient, and at least one hybrid containing the maternal allele and one hybrid containing the paternal allele of each MMR gene was isolated. Analysis of the nucleic acids from these hybrids revealed specific mutations in all ten patients (Table 1). In every case, an abnormality was found in a single allele of either hMSH2 or hMLH1. The nature of the abnormalities revealed why they had not been detected with the standard methods previously used for their analysis. Three cases were due to large deletions, encompassing six or seven exons. When corresponding nucleic acids from the cells of such patients are evaluated by any PCR-based method, only the wild type sequences from the unaffected parent would be amplified, leading to the false impression of normalcy (for example, case #1 in FIG. 3). Though Southern blotting can reveal deletions of one or a few exons in MMR, larger deletions are refractory to such blotting methods. In three cases (#4, 6, and 9), no transcript was generated from one allele, though the sequences of all exons and intron-exon borders from this allele were normal. Presumably, mutations deep within an intron or within the promoter of the gene were responsible. The absence of transcripts from one specific allele of these three patients was confirmed in at least three other converted hybrids from each patient. In four other cases, point mutations were found (Table 1). These mutations were not detected in the original sequence analyses because the signals from the mutant allele were not as robust as those from the wild type. Such asymmetry can be caused by instability of mutant transcripts due to nonsense mediated decay (36-38), or to nucleotide preferences of the polymerases in specific sequence contexts, and represents a common problem for both manual and automated sequencing methods (39). The conversion approach eliminates these problems because only one sequence can possibly be present at each position. A good example of this was provided by Warthin G (17). The mutation in this prototype kindred was an A to C transversion at a splice site. The signal from the mutant “C” in the sequencing ladder was not as intense as the wild type “A” (FIG. 4B). This mutation led to the use of a cryptic splice site 24 bp upstream of exon 4, and an under-represented transcript with a 24 base insertion (FIG. 4A). To demonstrate that this mutation had an effect at the protein level, we analyzed the hybrids by immunoblotting with specific antibodies (19). The hybrids containing the mutant allele did not make detectable levels of human hMSH2 protein, though they did synthesize normal levels of a control protein (FIG. 4C).

The results described above demonstrate that individual alleles of human chromosomes can be readily isolated upon fusion to mouse cells.

HNPCC provides a cogent demonstration of the power of the conversion approach because it is a common genetic disease that has been widely studied. In the last three years, for example, extensive analyses of the major MMR genes have been performed in 303 HNPCC kindreds from nine cohorts distributed throughout the world (25-34). Based on the fraction of such patients with characteristic microsatellite instability in their cancers (30-34), it can be estimated that 239 (78%) of the kindreds had germ-line mutations of mismatch repair genes. Yet MMR gene mutations were identified in only 127 (42%) of these 239 kindreds (25-34). Our cohort was similar, in that it was derived from a total of 25 kindreds, 22 of whom had tumors with microsatellite instability and presumptive MMR gene mutations. Of these 22, our initial analyses revealed mutations in only 12 (54%) (ref. 14 and unpublished data). Mutations of the other ten patients were only revealed upon conversion analysis, which thereby increased the sensitivity from 54% to 100%. The conclusion that virtually all cases of HNPCC associated with MSI are due to germline mutations of known MMR genes is consistent with recent immunohistochemical data demonstrating the absence of either MSH2 or MLH1 protein staining in the cancers from the great majority of HNPCC patients (40, 41). A corollary of these results is that the search for new human MMR genes should not based on the premise that a large fraction of HNPCC cases will prove attributable to such unknown genes.

The system described above can be applied to other genetic diseases in a straight forward manner. It should be emphasized that this approach is not a substitute for the many powerful methods currently available to search for specific mutations. Rather, conversion can be used to maximize the sensitivity of existing techniques. Converted nucleic acids provide the preferred substrates for such methods because of the higher signal to noise attainable and the inability of the wild type allele to mask or confound detection of the mutant allele. As DNA-based mutational assays are improved in the future, and progressive incorporate microarrays and other automatable features (42-44), the value of conversion-generated nucleic acids will correspondingly increase, significantly enhancing the effectiveness of genetic tests for hereditary disease.

### METHODS

Cell culture. Mouse embryonic fibroblasts were derived from MSH2-deficient mice (46) and transformed with adenovirus E1A and RAS oncogenes. HPRT-deficient subclones were selected by growing the fibroblasts in 10 μM 2-amino-6-mercaptopurine. Clones were maintained in Dulbecco's modified Eagle's Medium (DMEM) supplemented with 10% FCS and 10 μM 2-amino-6-mercaptopurine.

Cell fusion and the generation of hybrids. The patients were from kindreds with HNPCC as defined by the Amsterdam criteria (44); in no case was linkage analysis feasible due to the lack of a sufficient number of affected individuals. Microsatellite instability (MSI) in the cancers from these patients was determined through the markers recommended in ref. 45. 3×10⁶ E2 cells and 12×10⁶ lymphocytes cells were mixed, washed, and centrifuged twice in fusion medium (0.25 M D-sorbitol, 0.1 mM calcium acetate, 0.5 mM magnesium acetate, 0.1% Bovine Serum Albumin (BSA), pH 7) and resuspended in 640 μl fusion medium. The solution was pipetted into a cuvette (BTX cuvette electrode 470; BTX, San Diego). Cells were fused using a BTX ElectroCell Manipulator, model ECM200. The settings that yielded the greatest number of hybrids were: 30V (AC) for 22 seconds, followed by three 300V (DC) pulses of 15 μsec each. The cells from one fusion were plated into three 48-well plates (Costar) in DMEM supplemented with 10% FCS. After 24 hours, the medium was replaced by DMEM supplemented with 10% FCS, 0.5 mg/ml geneticin and 1×HAT (Life Technologies, Gaithersburg, Md.). The medium was changed after a week. Hybrid clones became visible two weeks after fusion and were expanded for another week prior to genotyping. From a single fusion, an average of 23±15 hybrid clones were obtained. The lymphocytes used for the experiments described here were derived from Epstein-Barr Virus infection of peripheral blood leukocytes, but it was found that freshly drawn lymphocytes could also be successfully fused and analyzed using identical methods.

Genotyping. Genotyping was performed as described (12). PCR products were separated on 6% denaturing gels and visualized by autoradiography. The microsatellite markers used were D2S1788 and D2S1360, D2S1384, D3S2406, D7S1824, and D16S3095, from chromosome 2p, 2q, 3p, 5q, 7q and 16q, respectively. Fluorescence in situ hybridization was performed as described previously (21).

PCR and sequencing. Polyadenylated RNA was purified and RT-PCR performed as described previously. Sequencing was performed using ABI Big Dye terminators and an ABI 377 automated sequencer. All primers used for amplification and sequencing will be made available through an internet site.

Statistical analysis. The number of hybrids containing none, both or a single allele of each chromosome tested were consistent with a multinomial distribution. Monte Carlo simulations were used to estimate the number of hybrids required to generate mono-allelic hybrids containing specific numbers of each chromosomes.

### REFERENCES AND NOTES

1. D. Ravine, Journal of Inherited Metabolic Disorders 22, 503 (1999); R. G. Cotton, Clin Chem Lab Med 36, 519 (1998)

2. F. J. Couch and B. L. Weber, in The Genetic Basis of Human Cancer, B. Vogelstein and K. W. Kinzler, Eds. (McGraw-Hill, N.Y., 1998), pp. 537-563; K. W. Kinzler, B. Vogelstein, Cell 87, 159 (1996).

3. T. P. Dryja, E. C. Berson Invest Ophthalmol Vis Sci 36:1197 (1995); G. C. Black, I. W. Craig, Mol Genet Med 4, 1 (1994); Inglehearn, Eye 12, 571 (1998).

4. B. Zoller, P. Garcia de Frutos, A. Hillarp, B. Dahlback, Haematologica 84, 59 (1999); H. G. Drexler, Leukemia 12, 845 (1998); M. Lawler, Radiat Oncol Investig 5, 154 (1997).

5. J. B. Martin, Science 262, 674 (1993); U. Muller, M. B. Graeber, G. Haberhausen, A. Kohler, J. Neurol Sci 124, 199 (1994); S. Sorbi, Aging (Milano) 5, 417 (1993).

6. C. E. Seidman, J. G. Seidman, Basic Res Cardiol 93, 13 (1998); M. T. Keating, M. C. Sanguinetti, Science 272, 681 (1996).

7. D. Patterson, D. V. Carnright, Somatic Cell Genet 3, 483 (1977); J. Groden, et al., Cell 66, 589 (1991); J. M. Gabriel et al., Proc Natl Acad Sci USA 95, 14857 (1998).

8. N. Papadopoulos, F. S. Leach, K. W. Kinzler, B. Vogelstein, Nature Genetics 11, 99 (1995); S. J. Laken et al., Proc Natl Acad Sci USA 96, 2322 (1999).

9. H. Harris, J Cell Sci 79, 83 (1985); M. J. Anderson, E. J. Stanbridge, FASEB J 7, 826(1993).

10. E2 cells were derived from mouse embryonic fibroblasts derived from MSH2-deficient mice (generously provided by T. Mak) and transformed with adenovirus E1A and RAS oncogenes. HPRT-deficient subclones were selected by growing the fibroblasts in 1 μM 2-amino-6-mercaptopurine. Clones were maintained in Dulbecco's modified Eagle's Medium (DMEM) supplemented with 10% FCS and 10 μM 2-amino-6-mercaptopurine.

11. 3×10⁶ lymphocytes cells were mixed, washed, and centrifuged twice in fusion medium (0.25 M D-sorbitol, 0.1 mM calcium acetate, 0.5 mM magnesium acetate, 0.1% Bovine Serum Albumin (BSA), pH 7) and resuspended in 640 μl fusion medium. The solution was pipetted into a cuvette (BTX cuvette electrode 470; BTX, San Diego). Cells were fused using a BTX Electro Cell Manipulator, model ECM200. The settings that yielded the greatest number of hybrids were: 30V (AC) for 22 seconds, followed by three 300V (DC) pulses of 15 μsec each. The cells from one fusion were plated into three 48-well plates (Costar) in DMEM supplemented with 10% FCS. After 24 hours, the medium was replaced by DMEM supplemented with 10% FCS, 0.5 mg/ml geneticin and 1×HAT (Life Technologies, Gaithersberg, Md.). The medium was changed after a week. Hybrid clones became visible two weeks after fusion and were expanded for another week prior to genotyping. The lymphocytes used for the experiments described here were derived from Epstein-Barr virus infection of peripheral blood leukocytes, but we found that freshly drawn lymphocytes could also be successfully fused and analyzed using identical methods

12. Genotyping was performed as described in F. S. Leach et al., Cell 75, 1215 (1993). PCR products were separated on 6% denaturing gels and visualized by autoradiography. The microsatellite markers used were D2S1788, D2S13360, D3S2406, D7S1824, and D16S3095, from chromosomes 2p, 2q, 3p, 5q, and 16q, respectively.

13. The numbers of hybrids containing none, both, or a single allele of each chromosome tested were consistent with a multinomial distribution. Monte Carlo simulations were used to estimate the numbers of hybrids required to generate mono-allelic hybrids containing specific numbers of chromosomes.

14. Polyadenylated RNA was purified and RT-PCR performed as described in B. Liu et al., Nat Medicine 2, 169 (1996).

15. C. R. Boland, Am. J. Dig. Dis 23, 25s-27s (1978); C. R. Boland, West J. Med. 139, 351 (183).

16. C. R. Boland, in The Genetic Basis of Human Cancer, B. Vogelstein and K. W. Kinzler, Eds (McGraw-Hill, N.Y., 1998), pp. 333-346.

17. A. S. Warthin, Archives of Internal Medicine 12, 546 (1913; H. T. Lynch and A. J. Krush, Cancer 27, 1505 (1971).

18. Sequencing was performed using ABI Big Dye terminators and an ABI 377 automated sequencer. All primers used for amplification and sequencing will be made available through a Science internet site.

19. Cytoplasmic extracts of cells hybrids were separated by electrophoresis through SDS-polyacrylamide gels and immunoblotted with antibodies specific for human hMSH2 (#NA26, Calbiochem), human hMLH1 (#13271A, Pharmingen), or β-Tubulin (#N357, Amersham).

20. D. R. Cox, Cytogenet Cell Genet 59, 80 (1992); M. A. Walter, D. J. Spillett, P. Thomas, J. Weissenbach, P. N. Goodfellow, Nat Genet 7, 22 (1994); R. J. Leach, P. O'Connell, Adv Genet 33, 63 (1995).

21. C. Lengauer, K. W. Kinzler, B. Vogelstein, Nature 386, 623 (1997).

22. M. Chee et al., Science 274, 610 (1996).

23. McDaniel, L. D., Legerski, R., Lehmann, A. R., Friedberg, E. C. & Schultz, R. A. Hum Mutat 10, 317-21 (1997)

24. Schultz, R. A., Saxon, P. J., Glover, T. W. & Friedbert, E. C. Proc Natl Acad Sci USA 84,4176-9 (1987).

25. Syngal, S. et al. JAMA 282, 247-53 (1999).

26. Bapat, B. V. et al.. Hum Genet 104, 167-76 (1999).

27. Wijnen, J. T. et al.. N Engl J Med 339, 511-8 (1998).

28. Wang, Q. et al. Hum Genet 105, 79-85 (1999)

29. Holmberg, M. et al. Hum Mutat 11, 482 (1998).

30. Bai, Y. Q. et al. Int J Cancer 82, 512-5 (1999).

31. Wijnen, J. Et al. Am J Hum Genet 61, 329-35 (1997).

32. Heinimann, K. et al. Cancer 85, 2512-8 (1999).

33. De Leon, M. P. et al. Gut 45, 32-8 (1999).

34. Lamberti, C. et al. Gut 44, 839-43 (1999).

35. Wijnen, J. et al. Nat Genet 20, 326-8 (1998).

36. Culbertson, M. R. Trends Genet 15, 74-80 (1999).

37. Frischmeyer, P. A. & Dietz, H. C.. Hum Mol Genet 8, 1893-900 (1999).

38. Ruiz-Echevarria, M. J., Czaplinski, K. & Peltz, S. W.. Trends Biochem Sci 21, 433-8 (1996).

39. Ahrendt, S. A. et al. Proc Natl Acad Sci USA 96 7382-7 (1999).

40. Marcus, V. A. et al. Am J Surg Pathol 23 1248-55 (1999).

41. Cawkwell, L. et al. Gut 45, 409-15 (1999).

42. Eng, C. & Vijg, J. Nat Biotechnol 15, 422-6 (1997).

43. Hacia, J. G. & Collins, F. S.. J Med Genet 36, 730-6 (1999).

44. Vasen, H. F., Mecklin, J. P., Khan, P. M. & Lynch, H. T. Anticancer Res 14 1661-4 (1994).

45. Boland, C. R. et al. Cancer Res 58, 5248-57 (1998).

46. Reitmair, A. H. et al.. Nat Genet 11, 64-70 (1995).

1. A method of identifying a personalized medical intervention for a non-rodent individual predisposed to or having a disorder associated with at least one polymorphic marker in at least one gene or in at least one intergenic region, comprising the steps of:
(a) fusing cells of the non-rodent individual to rodent cell recipients to form non-rodent/rodent cell hybrids;
(b) selecting for fused cell hybrids by selecting for a first selectable marker contained on a rodent chromosome and for a second selectable marker contained on a first non-rodent individual chromosome, to form a population of fused cell hybrids;
(c) detecting among the population of fused cell hybrids a subset of hybrids which are haploid for a second non-rodent individual chromosome which is not the same chromosome as the first non-rodent individual chromosome and which was not selected;
(d) analyzing said subset of hybrids to detect a polymorphic marker in the at least one gene, in a product of the gene, or in the intergenic region, wherein the gene or intergenic region resides on the second non-rodent individual chromosome; and
(e) selecting a medical intervention based on identity of the gene or intergenic region.

2. The method of claim 1 wherein the polymorphic marker is a single nucleotide polymorphism.

3. The method of claim 1 wherein the polymorphic marker is a microsatellite marker.

4. The method of claim 1 wherein the polymorphic marker is a plurality of polymorphic markers on the second non-rodent individual chromosome.

5. The method of claim 1 wherein the polymorphic marker is a mutation.

6. The method of claim 1 wherein selection of the medical intervention is based on the identity of the polymorphic marker.

7. The method of claim 1, further comprising the step of providing the medical intervention to the non-rodent individual.

8. The method of claim 1 wherein an mRNA product of the gene is analyzed in the subset of hybrids.

9. The method of claim 1 wherein a protein product of the gene is analyzed in the subset of hybrids.

10. The method of claim 1 wherein the gene is analyzed in the subset of hybrids.

11. The method of claim 1 wherein the intergenic region is analyzed in the subset of hybrids.

12. The method of claim 1 wherein the non-rodent individual is a human.

13. The method of claim 1 wherein the non-rodent individual is a dog.

14. The method of claim 1 wherein the subset of hybrids is analyzed to detect a plurality of polymorphic markers.

15. The method of claim 1 wherein the subset of hybrids is analyzed to detect polymorphic markers in at least two different genes or in at least two different intergenic regions.

16. The method of claim 1 wherein the polymorphic marker predisposes the individual to the disorder.

17. The method of claim 16 wherein the medical intervention is a prophylactic intervention.

18. The method of claim 1 wherein the polymorphic marker is causally related to the disorder.

19. The method of claim 1 wherein the polymorphic marker is associated with responsiveness to a drug and wherein the medical intervention is administration of the drug.

20. The method of claim 1 wherein the polymorphic marker is associated with resistance to a first drug useful for treating the disorder and wherein the medical intervention is administration of a second drug useful for treating the disorder.

21. A method of identifying a non-rodent individual as eligible to participate in a clinical trial to study the efficacy of a medical intervention, comprising the steps of:
(a) fusing cells of the non-rodent individual to rodent cell recipients to form non-rodent/rodent cell hybrids;
(b) selecting for fused cell hybrids by selecting for a first selectable marker contained on a rodent chromosome and for a second selectable marker contained on a first non-rodent chromosome, to form a population of fused cell hybrids;
(c) detecting among the population of fused cell hybrids a subset of hybrids which are haploid for a second non-rodent chromosome which is not the same chromosome as the first non-rodent chromosome and which was not selected;
(d) analyzing said subset of hybrids to detect a polymorphic marker in a gene, in a product of the gene, or in an intergenic region, wherein the gene or intergenic region resides on the second non-rodent chromosome; and
(e) identifying the non-rodent individual as eligible to participate in the clinical trial based on the presence, absence, or identity of the polymorphic marker which is detected.

22. The method of claim 21 wherein the polymorphic marker is a single nucleotide polymorphism.

23. The method of claim 21 wherein the polymorphic marker is a microsatellite marker.

24. The method of claim 21 wherein the polymorphic marker is a plurality of polymorphic markers on the second non-rodent individual chromosome.

25. The method of claim 21 wherein the polymorphic marker is a mutation.

26. The method of claim 21 wherein the at least one gene encodes a protein suspected of affecting the efficacy of a potential therapeutic agent.

27. The method of claim 21 wherein the polymorphic marker predisposes the non-rodent individual to a disorder and wherein the medical intervention may be efficacious to prevent, delay onset, or reduce severity of the disorder.

28. The method of claim 21 wherein the polymorphic marker is causally related to a disorder and wherein the medical intervention may be efficacious to treat the disorder.

29. The method of claim 21 further comprising the step of testing the non-rodent individual's response to the medical intervention.

30. The method of claim 21 wherein an mRNA product of the at least one gene is analyzed in the subset of hybrids.

31. The method of claim 21 wherein a protein product of the at least one gene is analyzed in the subset of hybrids.

32. The method of claim 21 wherein the at least one gene is analyzed in the subset of hybrids.

33. The method of claim 21 wherein the at least one intergenic region is analyzed in the subset of hybrids.

34. The method of claim 21 wherein the non-rodent individual is a human.

35. The method of claim 21 wherein the non-rodent individual is a dog.

36. The method of claim 21 wherein the subset of hybrids is analyzed to detect a plurality of polymorphic markers.

37. A method of identifying a polymorphic marker as associated with a first subpopulation of non-rodent individuals, comprising the steps of:
(a) fusing cells of a plurality of non-rodent individuals to rodent cell recipients to form a plurality of non-rodent/rodent cell hybrids;
(b) selecting for fused cell hybrids by selecting for a first selectable marker contained on a rodent chromosome and for a second selectable marker contained on a first non-rodent chromosome, to form a population of fused cell hybrids;
(c) detecting among the population of fused cell hybrids a subset of hybrids which are haploid for a second non-rodent chromosome which is not the same chromosome as the first non-rodent chromosome and which was not selected;
(d) analyzing said subset of hybrids to detect a polymorphic marker in a gene, in a product of the gene, or in an intergenic region, wherein the gene or intergenic region resides on the second non-rodent chromosome; and
(e) identifying the polymorphic marker as associated with the first subpopulation if the polymorphic marker is more prevalent in the first subpopulation and if the polymorphic marker is less prevalent in a second subpopulation of non-rodent individuals.

38. The method of claim 37 wherein the polymorphic marker is a single nucleotide polymorphism.

39. The method of claim 37 wherein the polymorphic marker is a microsatellite marker.

40. The method of claim 37 wherein the polymorphic marker is a set of polymorphic markers on the second non-rodent chromosome.

41. The method of claim 37 wherein the polymorphic marker is a mutation.

42. The method of claim 37 wherein an mRNA product of the gene is analyzed in the subset of hybrids.

43. The method of claim 37 wherein a protein product of the gene is analyzed in the subset of hybrids.

44. The method of claim 37 wherein the gene is analyzed in the subset of hybrids.

45. The method of claim 37 wherein the intergenic region is analyzed in the subset of hybrids.

46. The method of claim 37 wherein the non-rodent individuals are humans.

47. The method of claim 37 wherein the non-rodent individuals are dogs.

48. The method of claim 37 wherein the first subpopulation is a kindred.

49. The method of claim 37 wherein the subset t of hybrids is analyzed to detect a plurality of polymorphic markers.

50. The method of claim 37 wherein the subset of hybrids is analyzed to detect polymorphic markers in at least two different genes or in at least two different intergenic regions.

51. The method of claim 37 wherein the non-rodent individuals in the first subpopulation have a disorder.

52. The method of claim 51 wherein the polymorphic marker predisposes the individuals to the disorder.

53. The method of claim 51 wherein the polymorphic marker is causally related to the disorder.

54. A method of identifying a diagnostic test to be performed on a non-rodent individual predisposed to or having a disorder associated with a polymorphic marker in at least one gene or in at least one intergenic region, comprising the steps of:
(a) fusing cells of the non-rodent individual to rodent cell recipients to form non-rodent/rodent cell hybrids;
(b) selecting for fused cell hybrids by selecting for a first selectable marker contained on a rodent chromosome and for a second selectable marker contained on a first non-rodent individual chromosome, to form a population of fused cell hybrids;
(c) detecting among the population of fused cell hybrids a subset of hybrids which are haploid for a second non-rodent individual chromosome which is not the same chromosome as the first non-rodent individual chromosome and which was not selected;
(d) analyzing said subset of hybrids to detect a polymorphic marker in the at least one gene, in a product of the at least one gene, or in the at least one intergenic region, wherein the at least one gene or intergenic region resides on the second non-rodent individual chromosome; and
(e) identifying a diagnostic test based on the presence, absence, or identity of the polymorphic marker which is detected.

55. The method of claim 54 further comprising the step of performing the diagnostic test.

56. The method of claim 54 wherein the polymorphic marker is a single nucleotide polymorphism.

57. The method of claim 54 wherein the polymorphic marker is a microsatellite marker.

58. The method of claim 54 wherein the polymorphic marker is a plurality of polymorphic markers on the second non-rodent individual chromosome.

59. The method of claim 54 wherein the polymorphic marker is a mutation.

60. The method of claim 54 wherein selection of the diagnostic test is based on the detection of a particular third polymorphic marker.

61. The method of claim 54 wherein an mRNA product of the at least one gene is analyzed in the subset of hybrids.

62. The method of claim 54 wherein a protein product of the at least one gene is analyzed in the subset of hybrids.

63. The method of claim 54 wherein the gene is analyzed in the subset of hybrids.

64. The method of claim 54 wherein the intergenic region is analyzed in the subset of hybrids.

65. The method of claim 54 wherein the non-rodent individual is a human.

66. The method of claim 54 wherein the non-rodent individual is a dog.

67. The method of claim 54 wherein the subset of hybrids is analyzed to detect a plurality of polymorphic markers.

68. The method of claim 54 wherein the subset of hybrids is analyzed to detect polymorphic markers in at least two different genes or in at least two different intergenic regions.

69. The method of claim 54 wherein the polymorphic marker predisposes the individual to the disorder.

70. The method of claim 54 wherein the polymorphic marker is causally related to the disorder.
